(19) United States
(12) Patent Application Publication
(10) Pub. No.: US 2024/0008479 A1
(43) Pub. Date: Jan. 11, 2024

(54) ACTIVE COATING BASED ON PICKERING EMULSIONS

(71) Applicant: The State of Israel, Ministry of Agriculture & Rural Development, Agricultural Research Organization, Rishon Lezion (IL)

(72) Inventors: Guy MECHREZ, Adi (IL); Karthik Ananth MANI, Rishon Lezion (IL); Aviv DOMBROVSKY, Rishon Lezion (IL)

(21) Appl. No.: 18/371,366

(22) Filed: Sep. 21, 2023

Related U.S. Application Data
(63) Continuation of application No. PCT/IL2022/050330, filed on Mar. 24, 2022.
(60) Provisional application No. 63/165,879, filed on Mar. 25, 2021.

(51) Int. Cl. A01N 25/04; A01N 31/08; A01P 7/04; A01N 25/24

(52) U.S. Cl. CPC A01N 25/04; A01N 31/08; A01P 7/04; A01N 25/24

(57) ABSTRACT

A composition comprising an emulsion comprising a plurality of core-shell particles is provided. Furthermore, an insecticidal composition and use thereof so as to prevent or arrest growth of an insect or aphid is also provided. Further, a method for preventing or inhibiting growth of an insect or aphid on a plant or within the area under cultivation is provided.

### CROSS-REFERENCE TO RELATED APPLICATIONS

This application is a Bypass Continuation of PCT Patent Application No. PCT/IL2022/050330 having International filing date of Mar. 24, 2022, which claims the benefit of priority of U.S. Provisional Patent Application No. 63/165,879, titled “ACTIVE COATING BASED ON PICKERING EMULSIONS”, filed Mar. 25, 2021. The contents of both applications are incorporated herein by reference in their entirety.

### FIELD OF THE INVENTION

The present invention is in the field of Pickering emulsions.

### BACKGROUND OF THE INVENTION

Pickering emulsions are typically known as emulsions of any type, for example oil-in-water or water-in-oil, stabilized by solid particles in place of surfactants. Pickering emulsions are stabilized by nanoparticles (NPs) that are self-assembled typically at the oil-water interface and acts as a physical barrier.

### SUMMARY OF THE INVENTION

In one aspect of the invention, there is a composition comprising an emulsion, the emulsion comprising a major phase and a plurality of core-shell particles, wherein: each core-shell particle comprises a core comprising an aqueous solution, and a shell comprising hydrophobic metal oxide nanoparticles stabilizing the core; the major phase comprises an oil; and wherein a weight per weight (w/w) concentration of the hydrophobic metal oxide nanoparticles within the composition is between 0.001 and 10%.

In one embodiment, the shell further comprises a hydrophilic polymer in contact with the hydrophobic metal oxide nanoparticles, and wherein the hydrophilic polymer is water miscible or water dispersible.

In one embodiment, a w/w ratio between the hydrophilic polymer and the hydrophobic metal oxide nanoparticles is between 5:1 and 1:5.

In one embodiment, the hydrophilic polymer comprises a polyanion, a polycation, a polyol, including any mixture or a copolymer thereof.

In one embodiment, hydrophobic metal oxide nanoparticles comprises chemically modified metal oxide nanoparticles.

In one embodiment, chemical modification comprises any of (C1-C20) alkyl, (C1-C4) alkylsilyl, phenyl, thiol group, vinyl, fluoroalkyl, haloalkyl, halogen, epoxy, a cycloalkane, an alkene, a haloalkene, an alkyne, an ether, a silyl group, a siloxane group, and a thioether or any combination thereof.

In one embodiment, metal oxide comprises nanoclay, SiO2, TiO2, Al2O3, Fe2O3, ZnO, and ZrO or any combination thereof.

In one embodiment, core-shell particle has a diameter of 0.5 μm to 500 μm.

In one embodiment, the shell has a thickness of 10 nm to 100 μm.

In one embodiment, hydrophilic polymer comprises any one of a polyacrylate, carboxymethyl cellulose (CMC), alginate, pectin, chitosan, polymetacrylate, polyvinyl alcohol (PVA), or any copolymer or a combination thereof.

In one embodiment, a w/w concentration of the hydrophilic polymer within the composition is between 0.001 and 15%.

In one embodiment, a w/w ratio between the major phase and the aqueous solution within the composition is between 1:100 and 100:1.

In one embodiment, the oil comprises a vegetable oil, a mineral oil, and a lipid, or any combination thereof.

In one embodiment, hydrophilic polymer is immiscible with the oil.

In one embodiment, core-shell particles have a spherical shape, a quasi-spherical shape, a quasi-elliptical sphere, an irregular shape, or any combination thereof.

In one embodiment, composition comprises between 0.1 to 5% of an active agent selected from the group comprising a pesticide, a herbicide, and insecticide, or a combination thereof.

In one embodiment, active agent comprises an essential oil.

In one embodiment, emulsion comprises a Pickering emulsion, a water-in-oil emulsion, an oil-in-water emulsion or any combination thereof.

In one embodiment, composition has adhesiveness to a plant, a part of a plant, a plant tissue, a leaf, or any combination thereof.

In another aspect, there is an insecticidal composition comprising an effective amount of the composition of the invention.

In one embodiment, the insecticidal composition optionally comprises an agriculturally acceptable carrier.

In one embodiment, the hydrophobic metal oxide nanoparticle comprises (C1-C4) alkylsilyl modified silica, wherein the hydrophilic polymer comprises polyacrylate, and wherein the oil comprises plant oil.

In one embodiment, the insecticidal composition is for use as an insecticide.

In another aspect, there is a method for controlling a pest or reducing growth thereof, comprising applying an effective amount of the insecticidal composition of the invention to at least a portion of a plant, or an area under cultivation infested with the pest, thereby controlling or reducing growth of the pest.

In one embodiment, insecticidal composition is characterized by adhesiveness to at least a part of the plant.

In one embodiment, plant comprises a cultivating plant or a part thereof.

In one embodiment, pest is selected from an insect and an aphid.

In one embodiment, applying comprises any of immersion, coating, irrigating, dipping, spraying, fogging, scattering, painting, injecting, or any combination thereof.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 Presents a schematic illustration of a W/O Pickering emulsion;

FIGS. 2A-2D are images presenting confocal microscopy analysis of W/O Pickering emulsion (Canola oil:water) samples, showing the major oil phase stained by Nile red dispersing the particles of the invention comprising an aqueous core. FIGS. 2 A-B Images of the emulsion based on 1% Silica, canola oil:water (1:1), FIGS. 2C-D: Images of the emulsion based on 1% Silica, canola oil:water (1:1), 0.2% Carvacol, 0.2% Thymol.

FIGS. 3A-3C are images presenting confocal microscopy analysis of O/W Pickering emulsion (Canola oil:water). Confocal micrographs of oil in water Pickering emulsion based on 1% silica, canola oil and 0.5% Na-polyacrylate (SPA) in water. a) 1% silica, canola oil and 0.5% SPA in water (2:8), b) 1% silica, canola oil and 0.5% SPA in water (3:7), c) 1% silica, canola oil and 0.5% SPA in water (4:6). The emulsions were labelled with Nile red.

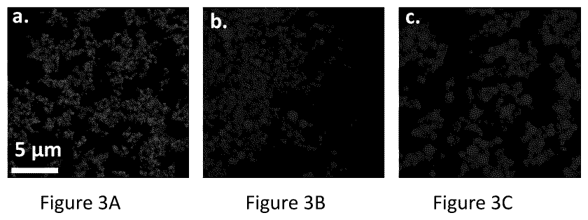

FIG. 4 is a graph presenting rheology analysis of w/o Pickering emulsion (Canola oil:water with and without active materials).

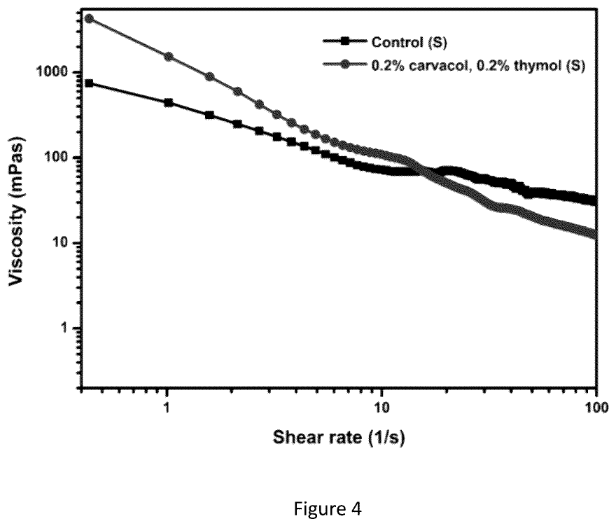

FIG. 5 is a graph presenting rheology analysis of O/W Pickering emulsion (Canola oil:water).

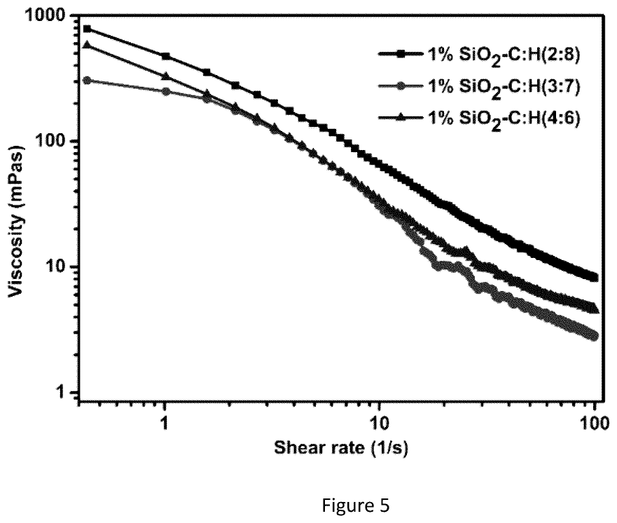

FIGS. 6A-6C are images presenting insect entrapment on the whole plant (FIG. 6A), front side of the leaf (FIG. 6B) and back side of the leaf (FIG. 6C), at day 7 after application of an exemplary O/W emulsion of the invention on chili pepper plants. The exemplary O/W emulsion is based on 1% Silica, SPA, and palm oil:water (2:8-3:7).

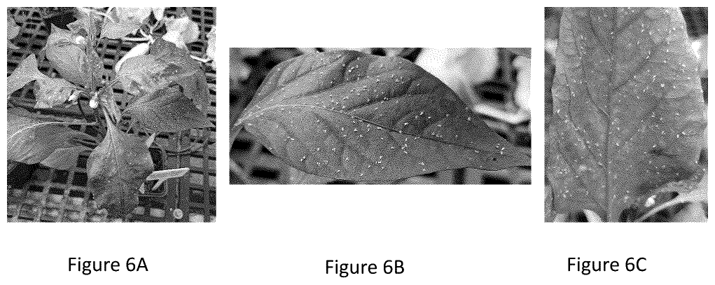

### DETAILED DESCRIPTION OF THE INVENTION

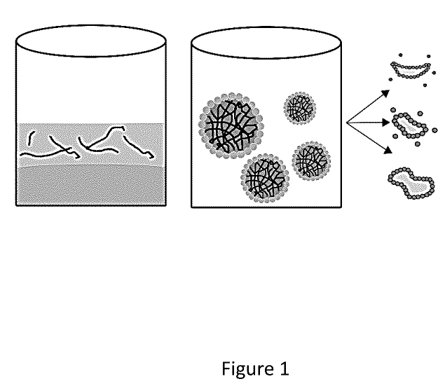

According to some embodiments, the present invention provides a composition comprising an emulsion comprising a plurality of particles. In some embodiments, the composition comprises a water-in-oil (W/O) Pickering emulsion. Reference is made to FIG. 1, which presents a schematic illustration of a W/O Pickering emulsion, according to some embodiments of the present invention. In some embodiments, the composition comprises an oil-in-water (O/W) Pickering emulsion. The emulsions according to the present invention comprises particles comprising a shell of nanoparticles and a core encapsulating a polymer. In some embodiments, the emulsions are used as active coatings.

According to some embodiments, the present invention provides a composition comprising an emulsion comprising a plurality of particles, the particles having a particle size (or diameter for substantially spherical particles) of 0.5 μm to 500 μm, comprising a shell having a thickness of 10 nm to 100 μm, and comprising hydrophobic inorganic nanoparticles. In some embodiments, the plurality of particles are dispersed within a major phase (e.g., on oil phase or an aqueous solution).

In some embodiments, the shell is a single layer shell. In some embodiments, the particles are in the interface of a major phase and a minor phase, wherein the emulsion is stabilized by the nanoparticles. In some embodiments, the composition of the invention and/or particles further comprise a polymer. In some embodiments, the polymer is a hydrophilic polymer. In some embodiments, the polymer is located in the shell of the particles. In some embodiments, the polymer binds the nanoparticles. In some embodiments, the polymer stabilizes the nanoparticles and/or stabilizes the shell. In some embodiments, the polymer contributes to the physical stability of the shell. In some embodiments, the polymer provides adhesiveness to the particles of the composition/emulsion. In some embodiments, adhesiveness refers to the adhesiveness of the particles of the composition/emulsion to a plant or a plant part (such as a leaf, including front side and/or back site of the leaf). In some embodiments, adhesiveness refers to the tackiness of the composition upon applying thereof to the plant and/or plant part (e.g., thereby obtaining a tacky coating layer on at least one surface of the plant and/or plant part).

In some embodiments, the particles are characterized by a core encapsulating an aqueous solution. In some embodiments, the core of the particles comprises an aqueous phase and the hydrophilic polymer. In some embodiments, the core of the particles comprises an oil, or an oil phase. In some embodiments, the core of the particles further comprises the hydrophilic polymer and/or an active agent dissolved or dispersed within the aqueous phase or within the oil phase, respectively.

According to some embodiments, the present invention provides a coating composition. In some embodiments, the composition of the invention is an agricultural composition for application on a plant a part of a plant or area under cultivation. In some embodiments, the composition of the invention is an insecticidal composition. In some embodiments, there is a composition comprising a plant and/or a part thereof in contact with the composition of the invention comprising a plurality of core-shell particles, wherein the plurality of core-shell particles are in the form of a coating layer on the plant and/or on the plant part (e.g., leaf, stem and/or branch). In some embodiments, the particles enclose therewithin an active agent.

In some embodiments, the composition is substantially devoid of phytotoxicity. In some embodiments, the composition results in a coating layer on the plant and/or a part thereof upon application thereof on the plant surface and exposing to conditions sufficient for drying of the composition. In some embodiments, the coating layer is an adhesive layer. In some embodiments, the coating layer is characterized by adhesiveness to a pest (e.g., an insect, an aphid or both). In some embodiments, the coating layer is characterized by adhesiveness to a plant surface. In some embodiments, the coating layer is stably bound to a plant surface.

In some embodiments, the coating layer is characterized by a slow release, wherein the slow release of the active agent encapsulated within the core-shell particle.

### The Composition

In one aspect of the invention, there is a composition comprising an emulsion or dispersion. In some embodiments, the emulsion is an O/O Pickering emulsion. In some embodiments, the emulsion is a W/O Pickering emulsion. In some embodiments, the emulsion is an O/W Pickering emulsion.

In some embodiments, the composition comprises an emulsion or dispersion, comprising a plurality of particles. In some embodiments, the particles are in the form of droplets. In some embodiments, the composition further comprises an active agent (a pesticide, a pest attracting agent, an aphid attracting agent, etc.). In some embodiments, the composition further comprises a tackifier. Various tackifiers are well-known in the art, such as glycine.

As used herein, the term “Pickering emulsion” refers to an emulsion that utilizes solid particles as a stabilizer to stabilize droplets of a substance, in a dispersed phase in the form of droplets dispersed throughout a continuous phase.

As used herein, the term “emulsion” refers to a combination of at least two fluids, where one of the fluids is present in the form of droplets in the other fluid. The term “emulsion” includes microemulsions.

As used herein, the term “fluid” refers to a substance that tends to flow and to conform to the outline of its container, i.e., a liquid, a gas, a viscoelastic fluid, etc. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids. In some cases, the droplets may be contained within a carrier fluid, e.g., a liquid.

In some embodiments, the composition of the invention is characterized by a viscosity between 10 and 10.000 cP, between 10 and 1000 cP, between 10 and 100 cP, between 100 and 10.000 cP, between 100 and 1.000 cP, including any range between, wherein the viscosity is measured at a shear rate of 1/s (at a temperature about 25° C.)

In another aspect, there is emulsion comprising a major phase and a plurality of core-shell particles, wherein each core-shell particle comprises a core comprising an aqueous solution, and a shell comprising hydrophobic metal oxide nanoparticles stabilizing the core; the major phase comprises an oil; and wherein a weight per weight (w/w) concentration of the hydrophobic metal oxide nanoparticles within the composition is between 0.001 and 10%. In some embodiments, the core-shell particles are stably dispersed within the emulsion. In some embodiments, the emulsion is stable (e.g., devoid of aggregation, phase separation, release of the core content) for a time period of at least 1 day, at least 1 week, at least 1 month, at least 1 year, including any range between. In some embodiments, the stable emulsion is characterized by substantially constant (e.g., deviation of less than 30%) particle size over a time period ranging from 1 day to 1 month (m), from 1 m to 2 m, from 2 m to 4 m, from 4 m to 6 m, from 6 m to 8 m, from 8 m to 10 m, from 10 m to 12 m, including any range between.

In some embodiments, the emulsion further comprises a hydrophilic polymer. In some embodiments, the hydrophilic polymer and the hydrophobic metal oxide nanoparticles stabilize the emulsion and/or the core-shell particles. In some embodiments, the terms “core-shell particle” and “particle” are used herein interchangeably.

In some embodiments, the hydrophilic polymer of the invention is water miscible or water dispersible. In some embodiments, the hydrophilic polymer is characterized by an HLB of between 6 and 18, between 6 and 10, between 10 and 12, between 12 and 15, between 15 and 18, including any range between. In some embodiments, the hydrophilic polymer of the invention is immiscible with the major phase of the invention.

In some embodiments, the shell of the core-shell particle comprises a plurality of hydrophobic metal oxide nanoparticles. In some embodiments, the shell of the core-shell particle comprises a plurality of hydrophobic metal oxide nanoparticles in contact with the hydrophilic polymer. In some embodiments, the hydrophilic polymer forms an intertwined network within the particle. In some embodiments, the hydrophilic polymer forms an intertwined network within the shell of the particle. In some embodiments, the hydrophilic polymer forms an intertwined network within the shell and/or within the core of the particle. In some embodiments, the hydrophilic polymer forms an intertwined network within the inner portion and/or within the outer portion of the shell, wherein the inner portion faces the core and the outer potion of the shell face the ambient (e.g., the major phase). In some embodiments, the hydrophilic polymer has an amorphous structure within the particle. In some embodiments, the hydrophilic polymer has an amorphous structure within the particle shell. In some embodiments, the hydrophobic metal oxide nanoparticles are in embedded within or bound to the polymeric network. In some embodiments, the hydrophobic metal oxide nanoparticles are bound to a surface (e.g., top or bottom) of the polymeric network.

In some embodiments, the hydrophilic polymer and the hydrophobic metal oxide nanoparticles comprise up to 80%, up to 85%, up to 90%, up to 92%, up to 95%, up to 97%, up to 99%, up to 98%, up to 96% w/w of the particle's shell. In some embodiments, the hydrophilic polymer and the hydrophobic metal oxide nanoparticles comprise up to 80%, up to 85%, up to 90%, up to 92%, up to 95%, up to 97%, up to 99%, up to 98%, up to 96% w/w of the dry matter content of the composition of the invention.

In some embodiments, the particle has a spherical geometry or shape. In some embodiments, the particle has an inflated or a deflated shape. In some embodiments, a plurality of particles is devoid of any characteristic geometry or shape. In some embodiments, the plurality of particles are substantially spherically shaped. In some embodiments, a volume of the core comprises at most 95%, at most 90%, at most 85%, at most 80%, at most 75%, at most 70%, at most 60%, at most 50%, at most 40%, at most 30%, at most 20% v/v of a fluid (such as an aqueous solution, and optionally the hydrophilic polymer and/or an active agent).

In some embodiments, the plurality of particles is characterized by a particle size between 0.5 μm and 500 μm, 1 μm to 100 μm, 5 μm to 100 μm, 10 μm to 100 μm, 50 μm to 100 μm, 1 μm to 80 μm, 10 μm to 80 μm, 50 μm to 80 μm, 10 μm to 50 μm, 80 μm to 100 μm, 100 μm to 200 μm, 200 μm to 300 μm, 300 μm to 400 μm, 400 μm to 500 μm, 1 μm to 10 μm, 5 μm to 10 μm, 1 μm to 50 μm, 10 μm to 50 μm, 5 μm to 50 μm, or 1 μm to 5 μm, including any range or value therebetween. In some embodiments, the plurality of particles is characterized by a particle size between 500 nm and 10 μm, between 500 nm and 1 μm, between 500 nm and 5 μm, between 1 μm and 10 μm, between 1 μm and 5 μm, between 5 μm and 10 μm, including any range or value therebetween.

In some embodiments, the particle size of the particles described herein, represents an average particle size. In some embodiments, the size of the particle described herein represents an average size of a plurality of particles. In some embodiments, the average size of at least e.g., 10%, 20%, 30%, 40%, 50%, 60%, 70%, 80%, 90%, or 95% of the particles, ranges from: 5 μm to 50 μm, 1 μm to 50 μm, 5 μm to 10 μm, including any range therebetween. In some embodiments, the particle size of the particle described herein, is a dry particle size (i.e., a particle size of isolated dried particles). In some embodiments, a plurality of the particles has a uniform size. By “uniform” or “homogenous” it is meant to refer to size distribution that varies within a range of less than e.g., +60%, +50%, +40%, ±30%, +20%, or +10%, including any value therebetween.

In some embodiments, the particle is in a form of a colloidosome. In some embodiments, the particle is in a solid form. In some embodiments, the particle is in a form of a droplet.

In some embodiments, the droplets have a particle size of 1 μm to 100 μm, 5 μm to 100 μm, 10 μm to 100 μm, 50 μm to 100 μm, 1 μm to 80 μm, 10 μm to 80 μm, 50 μm to 80 μm, 1 μm to 10 μm, 5 μm to 10 μm, 1 μm to 50 μm, 10 μm to 50 μm, 5 μm to 50 μm, or 1 μm to 5 μm, including any range therebetween. In some embodiments, the droplets are characterized by a particle size between 500 nm and 10 μm, between 500 nm and 1 μm, between 500 nm and 5 μm, between 1 μm and 10 μm, between 1 μm and 5 μm, between 5 μm and 10 μm, including any range or value therebetween.

As used herein, the term “droplet” refers to an isolated portion of a first fluid that is surrounded by a second fluid. It is to be noted that a droplet is not necessarily spherical; but may assume other shapes as well, for example, depending on the external environment. In some embodiments, the droplet has a minimum cross-sectional dimension that is substantially equal to the largest dimension of the channel perpendicular to fluid flow in which the droplet is located. In some cases, the droplet may be a vesicle, such as a liposome, a colloidosome, or a polymerosome. The fluidic droplets may have any shape and/or size. Typically, monodisperse droplets are of substantially the same size. The shape and/or size of the fluidic droplets can be determined, for example, by measuring the average particle size or other characteristic dimension of the droplets. The “average particle size” of a plurality or series of droplets is the arithmetic average of the average particle sizes of each of the droplets. Those of ordinary skill in the art will be able to determine the average particle size or diameter (or other characteristic dimension) of a plurality or series of droplets, for example, using laser light scattering, microscopic examination, or other known techniques. The average particle size of a single droplet, in a non-spherical droplet, is the diameter of a perfect sphere having the same volume as the non-spherical droplet. In some embodiments, the average particle size of a droplet (and/or of a plurality or series of droplets) is, 5 μm to 100 μm, 5 μm to 50 μm, 1 μm to 50 μm, including any range therebetween. In some embodiments, the average particle size of a droplet is a wet particle size (i.e., a particle dimeter within a solution).

In some embodiments, the composition of the invention comprises an emulsion or dispersion, comprising a plurality of particles, having a particle size of 0.5 μm to 100 μm, the particles comprising a shell having a thickness of 5 nm to 100 nm, and comprising inorganic hydrophobic nanoparticles. In some embodiments, the shell has a thickness in the range of 5 nm to 50 nm, 15 nm to 50 nm, 30 nm to 50 nm, 1 nm to 50 nm, 2 nm to 50 μm, 5 μm to 10 μm, 10 nm to 50 nm, 5 nm to 30 nm, 15 nm to 30 nm, 1 nm to 20 μm, 2 nm to 20 nm, 5 nm to 20 nm, or 10 nm to 20 nm, including any range therebetween. In some embodiments, the shell thickness is quantified using scanning electron microscopy. In some embodiments, the composition comprises a solvent, selected from an aqueous solvent, a lipophilic organic solvent and a polar organic solvent or any combination thereof.

In some embodiments, the composition (e.g. an emulsion) comprises 0.01% to 10% (w/w), 0.05% to 10% (w/w), 0.09% to 10% (w/w), 0.1% to 10% (w/w), 0.5% to 10% (w/w), 0.9% to 10% (w/w), 1% to 10% (w/w), 5% to 10% (w/w), 0.01% to 9% (w/w), 0.05% to 9% (w/w), 0.09% to 9% (w/w), 0.1% to 9% (w/w), 0.5% to 9% (w/w), 0.9% to 9% (w/w), 1% to 9% (w/w), 5% to 9% (w/w), 0.01% to 5% (w/w), 0.05% to 5% (w/w), 0.09% to 5% (w/w), 0.1% to 5% (w/w), 0.5% to 5% (w/w), 0.9% to 5% (w/w), or 1% to 5% (w/w), of the particles, including any range therebetween.

In some embodiments, the particle comprises a core at least partially surrounded or enclosed by a shell, wherein the core and the shell are as described herein. In some embodiments, the inner portion of the shell is in contact with the core. In some embodiments, the inner portion is bound to the core. In some embodiments, the shell stabilizes the core. In some embodiments, the shell encapsulates the core.

In some embodiments, the shell has a thickness between 10 nm and 100 μm, between 10 and 100 nm, between 100 and 500 nm, between 500 nm and 1 μm, between 1 and 10 μm, between 10 and 20 μm, between 20 and 50 μm, between 50 and 70 μm, between 70 and 90 μm, between 90 and 100 μm, including any range therebetween.

In some embodiments, the shell comprises between 10% and 99%, between 10% and 20%, between 20% and 30%, between 30% and 50%, between 50% and 60%, between 60% and 70%, between 70% and 80%, between 80% and 90%, between 90% and 99% (w/w) of the hydrophobic metal oxide nanoparticles.

In some embodiments, the particle of the invention comprises between 1% and 90%, between 10% and 99%, between 10% and 20%, between 20% and 30%, between 30% and 50%, between 50% and 60%, between 60% and 70%, between 70% and 80%, between 80% and 90%, between 90% and 99% (w/w) of the hydrophilic polymer.

In some embodiments, the shell is devoid of a polymer, consisting essentially of hydrophobic metal oxide nanoparticles. In some embodiments, the shell is stabilized by the polymer and/or by the hydrophobic metal oxide nanoparticles. In some embodiments, the shell comprises the hydrophilic polymer bound to the hydrophobic metal oxide nanoparticles. In some embodiments, the hydrophobic metal oxide nanoparticles are adhered to the hydrophilic polymer. In some embodiments, the hydrophobic metal oxide nanoparticles are held together by the hydrophilic polymer. In some embodiments, a portion of the hydrophilic polymer enhances the stability of the shell.

In some embodiments, the inner portion of the shell is bound or in contact with the polymeric portion of the core. In some embodiments, the shell is bound or in contact with the polymeric portion of the core. In some embodiments, the inner portion of the shell, the outer portion of the shell or both comprise the hydrophilic polymer.

In some embodiments, the core comprises between 0.1% and 10%, between 0.1% and 1%, between 1% and 5%, between 5% and 10%, (w/w) of the hydrophilic polymer, including any range therebetween.

In some embodiments, the hydrophilic polymer is chemically stable (e.g., maintains at least 90% of its chemical structure) at a temperature of 100° C., of 80° C., of 90° C., of 70° C., of 60° C., of 50° C., of 40° C. including any range or value therebetween.

In some embodiments, the hydrophilic polymer is soluble in an aqueous solution, wherein soluble is at least 1 g/L, at least 5 g/L, at least 10 g/L, at least 30 g/L, at least 50 g/L, at least 100 g/L, including any range between.

In some embodiments, the hydrophilic polymer is dispersible in an aqueous solution.

In some embodiments, the hydrophilic polymer has an affinity to the hydrophobic metal oxide nanoparticles. In some embodiments, the hydrophilic polymer adheres to the hydrophobic metal oxide nanoparticles.

In some embodiments, the hydrophilic polymer comprises a polyanion, a polycation, a polyol, including any mixture or a copolymer thereof. In some embodiments, the polyol comprises PEG, PPG, a polyalcohol, a polysaccharide, or a combination or a copolymer thereof.

In some embodiments, the hydrophilic polymer comprises any one of a polyacrylate, an acrylate polymer (e.g., an alkyl ester of a polyacrylic acid), polyacrylic acid, carboxymethyl cellulose (CMC), alginate, alginic acid, pectin, chitosan, polymetacrylate, polyvinyl alcohol (PVA), including any salt, any polymer in a protonated form (e.g., carboxylic acid instead of carboxylate, a protonated amino group of chitosan, etc.), any copolymer and/or a combination thereof. In some embodiments, the hydrophilic polymer of the invention comprises a polyacrylate, a polymetacrylate, a polymethylmetacrylate including any salt, any copolymer or a combination thereof. In some embodiments, the hydrophilic polymer of the invention comprises a polyanion (e.g., a polyacrylate and/or a salt thereof).

In some embodiments, a w/w ratio between the hydrophilic polymer to the hydrophobic metal oxide nanoparticles is between 5:1 and 1:5, between 5:1 and 3:1, between 3:1 and 2:5, between 2:1 and 1:1, between 1:1 and 1:2, between 1:2 and 1:3, between 1:3 and 1:5, including any range between.

In some embodiments, the composition of the invention and/or the particle shell comprises a plurality of nanoparticles. In some embodiments, the nanoparticles are hydrophobic. In some embodiments, the outer surface of the nanoparticles is hydrophobic. In some embodiments, the nanoparticles comprise inorganic particles. In some embodiments, the nanoparticles comprise chemically modified inorganic particles. In some embodiments, the nanoparticles comprise inorganic particles having a chemical modification (e.g., a hydrophobic group attached thereto). In some embodiments, the nanoparticles are hydrophobic metal oxide nanoparticles.

In some embodiments, the hydrophobic metal oxide nanoparticles comprise a metal oxide. In some embodiments, the hydrophobic metal oxide nanoparticles are metal oxide-based particles. In some embodiments, the metal oxide is selected from the group consisting of silica, titanium oxide, clay, and any combination thereof.

In some embodiments, hydrophobic metal oxide nanoparticles are selected from fluoro-hydrophobic nanoparticles, silane-hydrophobic nanoparticles, or both.

Non-limiting examples of silane-hydrophobic nanoparticles include silane, methyl silane, linear alkyl silane, branched alkyl silane, aromatic silane, fluorinated alkyl silane, and dialkyl silane.

The term “silica” as used here refers to a structure containing at least the following the elements: silicon and oxygen. Silica may have the fundamental formula of SiO₂, or it may have another structure including SiₓO_{y} (where x and y can each independently be about 1 to 10). Additional elements including, but not limited to, carbon, nitrogen, sulfur, phosphorus, or ruthenium may also be used. Silica may be a solid particle, or it may have pores.

In some embodiments, the hydrophobic metal oxide nanoparticles comprise chemically modified metal oxide, wherein the metal oxide comprises nanoclay, SiO₂, TiO₂, Al₂O₃, Fe₂O₃, ZnO, and ZrO or any combination thereof.

In some embodiments, the chemical modification comprises any of (C1-C20)alkyl, (C1-C4)alkylsilyl, phenyl, thiol group, vinyl, fluoroalkyl, haloalkyl, halogen, epoxy, a cycloalkane, an alkene, a haloalkene, an alkyne, an ether, a silyl group, a siloxane group, and a thioether or any combination thereof. In some embodiments, the hydrophobic metal oxide nanoparticle comprises a silylated silica. In some embodiments, the hydrophobic metal oxide nanoparticle comprises C1-C4 alkyl-silylated silica, and/or C1-C20 alkyl silylated silica. In some embodiments, the hydrophobic metal oxide nanoparticle comprises C1 alkyl-silylated silica (e.g., Aerosil 812), or a C16 alkyl-silylated silica (e.g., Aerosil 816).

In some embodiments, the hydrophobic metal oxide nanoparticles are characterized by an average particle size of 1 nm to 900 nm. In some embodiments, the hydrophobic metal oxide nanoparticles are characterized by an average particle size of 2 nm to 20 nm, 2 nm to 10 nm, 2 nm to 50 nm, 2 nm to 30 nm, 2 nm to 40 nm, including any range therebetween. In some embodiments, the hydrophobic metal oxide nanoparticles are characterized by an average particle size of 2 nm to 600 nm, 2 nm to 550 nm, 2 nm to 520 nm, 2 nm to 500 nm, 2 nm to 480 nm, 2 nm to 450 nm, 2 nm to 400 nm, 2 nm to 350 nm, 2 nm to 300 nm, 2 nm to 250 nm, 2 nm to 200 nm, 2 nm to 150 nm, 2 nm to 100 nm, 5 nm to 600 nm, 10 nm to 600 nm, 15 nm to 600 nm, 20 nm to 600 nm, 40 nm to 600 nm, 50 nm to 600 nm, 100 nm to 600 nm, 5 nm to 500 nm, 10 nm to 500 nm, 15 nm to 500 nm, 20 nm to 500 nm, 40 nm to 600 nm, 50 nm to 500 nm, 100 nm to 500 nm, 5 nm to 400 nm, 10 nm to 400 nm, 15 nm to 400 nm, 20 nm to 400 nm, 40 nm to 400 nm, 50 nm to 400 nm, 100 nm to 400 nm, 5 nm to 50 nm, 5 nm to 40 nm, 2 nm to 50 nm, or 2 nm to 40 nm, including any range therebetween. In some embodiments, the size of at least 90% of the hydrophobic metal oxide nanoparticles varies within a range of less than ±25%, ±20%, ±15%, ±19%, 5%, including any value therebetween.

Herein throughout, the terms “nanoparticle”, “nano”, “nanosized”, and any grammatical derivative thereof, which are used herein interchangeably, describe a particle featuring a size of at least one dimension thereof (e.g., particle size, length) that ranges from about 1 nanometer to 100 nanometers. Herein throughout, “NP(s)” designates nanoparticle(s).

As used herein the terms “average” or “mean” size refer to particle size of the particles. The term “particle size” is art-recognized and is used herein to refer to either of the physical diameter (also termed “dry diameter”) or the hydrodynamic diameter. As used herein, the “hydrodynamic diameter” refers to a size determination for the composition in solution (e.g., aqueous solution) using any technique known in the art, e.g., dynamic light scattering (DLS).

In some embodiments, the dry diameter of the particles, as prepared according to some embodiments of the invention, may be evaluated using transmission electron microscopy (TEM) or scanning electron microscopy (SEM) imaging.

The particle(s) can be generally shaped as a sphere, incomplete-sphere, particularly the size attached to the substrate, a rod, a cylinder, a ribbon, a sponge, and any other shape, or can be in a form of a cluster of any of these shapes, or a mixture of one or more shapes. In some embodiments, the particle has a spherical shape, a quasi-spherical shape, a quasi-elliptical sphere, an irregular shape, or any combination thereof.

In some embodiments, the emulsion of the invention comprises a major phase dispersing the core-shell particles. In some embodiments, the major phase and a minor phase are liquids at a temperature between 1 and 90° C. In some embodiments, the major phase and a minor phase are immiscible liquids. In some embodiments, the major phase of the invention comprises an oil. In some embodiments, the major phase of the invention comprises an aqueous solution.

As used herein, the term “oil” refers to any suitable water-immiscible compound. In some embodiments, the oil is an oil that is liquid at room temperature (20° C.; 1013 mbar). In embodiments, the oil is selected from the group consisting of essential oils, vegetable oils, mineral oils, organic oils, lipids, fatty acids and/or esters thereof, and any water-immiscible liquids.

In some embodiments, the oil comprises a mineral oil, a hydrocarbon (e.g., C10-100 hydrocarbon), a fatty acid (saturated and/or unsaturated), a mono-, di-, triacylglycerols, a vegetable oil, a plant oil, a wax or any combination thereof.

In some embodiments, the plant oil is selected from the group consisting of: an olive oil, a canola oil, a triglyceride oil, a terpenoid oil, a citrus oil, a sunflower oil, a peanut oil, a soy oil, a rapeseed oil, a soybean oil, a palm oil, a cocoa butter, a rice bran oil, and limonene or any combination thereof.

In some embodiments, the major phase of the invention comprises a mineral oil and/or a plant oil (e.g., canola oil, sunflower oil, etc.). In some embodiments, the major phase of the invention comprises an aqueous solution and the minor phase comprises a mineral oil and/or a plant oil (e.g., canola oil, sunflower oil, etc.).

As used herein throughout, the term “polymer” describes an organic substance composed of a plurality of repeating structural units (backbone units) covalently connected to one another. In some embodiments, the thermoplastic polymer comprises a polyacrylate, polysiloxane, polyethylene, polyisocyanate, polyurethane, fluorinated polymer, perfluorinated polymer, Teflon, Teflon PTFE, polyvinylchloride, polydimethylsiloxane, polystyrene, polytetrafluoroethylene, or any combination thereof.

In some embodiments, the composition of the invention substantially (e.g., at least 90%, at least 93%, at least 95%, at least 97%, at least 99%, at least 99.9% by weight of the composition) comprises agriculturally acceptable ingredients. In some embodiments, the composition of the invention substantially (e.g., at least 90%, at least 93%, at least 95%, at least 97%, at least 99%, at least 99.9% by weight of the composition) consist of agriculturally acceptable ingredients. In some embodiments, the composition of the invention is substantially devoid of an organic solvent. In some embodiments, the composition of the invention is substantially devoid of an additional polymer, and/or additional nanoparticle.

In some embodiments, the hydrophobic metal oxide nanoparticle are in the interface between the major phase and a minor phase. In some embodiments, the major phase is a continuous phase. In some embodiments, a minor phase is a dispersed phase. In some embodiments, the term “minor phase” refers to the liquid within the core of the core-shell particles disclosed herein. In some embodiments, the term “major phase” refers to the liquid the core-shell particles are dispersed therewith.

In some embodiments, the major phase is a water phase. In some embodiments, the major phase is an oil phase. In some embodiments, the minor phase is a water phase. In some embodiments, the minor phase is an oil phase.

In some embodiments, the ratio of the major phase and the minor phase within the composition of the invention is 100:1 to 1:100 (w/w), 100:1 to 10:1 (w/w), 10:1 to 5:1 (w/w), 3:1 to 1:1 (w/w), 2:1 to 1:1 (w/w), 1:1 to 1:3 (w/w), 1:3 to 1:5 (w/w), 1:5 to 1:10 (w/w), 1:10 to 1:100 (w/w), including any range therebetween.

In some embodiments, the ratio of the major phase and the minor phase within the composition of the invention is 10:1 to 1:10 (w/w), 10:1 to 5:1 (w/w), 3:1 to 1:1 (w/w), 2:1 to 1:1 (w/w), 1:1 to 1:3 (w/w), 1:3 to 1:5 (w/w), 1:5 to 1:10 (w/w), 1 including any range therebetween.

In some embodiments, the ratio of the major phase (and the minor phase within the composition of the invention is 10:1 to 1:1 (w/w), 10:1 to 1.2:1 (w/w), 10:1 to 8:1 (w/w), 8:1 to 5:1 (w/w), 8:1 to 1:1 (w/w), 5:1 to 1:1 (w/w), 5:1 to 1.2:1 (w/w), 4:1 to 1:1 (w/w), 3:1 to 1:1 (w/w), or 2:1 to 1:1 (w/w), including any range therebetween. In some embodiments, the ratio of the major phase and the minor phase is 1:1 (w/w).

In some embodiments, a w/w concentration of the hydrophilic polymer within the composition is between 0.001 and 15%, between 0.001 and 0.01%, between 0.01 and 0.1%, between 0.1 and 0.5%, between 0.5 and 1%, between 1 and 3%, between 3 and 5%, between 5 and 15%, including any range therebetween.

In some embodiments, the composition of the invention comprises a plant oil as the major phase, a polyacrylate as the hydrophilic polymer, and C1-C20 alkylsilylated silica as the hydrophobic metal oxide nanoparticle. In some embodiments, the composition of the invention (e.g., emulsion) comprises a plant oil as the major phase, a polyacrylate as the hydrophilic polymer, and C1-C20 alkylsilylated silica as the hydrophobic metal oxide nanoparticle, wherein a w/w concentration of the polymer and of the hydrophobic metal oxide nanoparticle within the composition is between 0.1 and 2%. In some embodiments, the composition of the invention (e.g. emulsion) comprises a plant oil as the major phase, a polyacrylate as the hydrophilic polymer, and C1-C20 alkylsilylated silica as the hydrophobic metal oxide nanoparticle, wherein a w/w concentration of the polymer and of the hydrophobic metal oxide nanoparticle within the composition is between 0.1 and 2%, and wherein a volume per volume ratio between the major phase (or “oil”, as described herein) and the aqueous solution within the composition is between 2:1 and 1:2.

In some embodiments, the composition of the invention comprises an aqueous solution as the major phase, a plant oil and/or a mineral oil as the minor phase, a polyacrylate as the hydrophilic polymer, and C1-C20 alkylsilylated silica as the hydrophobic metal oxide nanoparticle. In some embodiments, the composition of the invention (e.g., emulsion) comprises a plant oil and/or a mineral oil as the minor phase, a polyacrylate as the hydrophilic polymer, and C1-C20 alkylsilylated silica as the hydrophobic metal oxide nanoparticle, wherein a w/w concentration of the polymer and of the hydrophobic metal oxide nanoparticle within the composition is between 0.1 and 2% including any range between. In some embodiments, the composition of the invention (e.g. emulsion) comprises a plant oil and/or a mineral oil as the minor phase, a polyacrylate as the hydrophilic polymer, and C1-C20 alkylsilylated silica as the hydrophobic metal oxide nanoparticle, wherein a w/w concentration of the polymer and of the hydrophobic metal oxide nanoparticle within the composition is between 0.1 and 2% including any range between, and wherein a volume per volume ratio between the major phase (or aqueous solution”) and the minor phase (or “oil”, as described herein) within the composition is between about 10:1 and 1.2:1, or between about 5:1 and 1.2:1, including any range between.

In some embodiments, the composition of the invention further comprises between 0.1% and 10%, between 0.1% and 1%, between 1% and 5%, between 5% and 10% w/w of the active agent. In some embodiments, the active agent is selected from the group comprising a pesticide, an herbicide, and insecticide, or a combination thereof.

In some embodiments, the minor phase (e.g., the core of the core-shell particle) comprises an active agent. In some embodiments, the major phase (e.g., oil) comprises an active agent. In some embodiments, the emulsion comprises an active agent dissolved in the minor and or major phase. In some embodiments, the core of the particles encapsulates an active agent.

In some embodiments, the active agent has a boiling temperature of more than 40° C., of more than 50° C., of more than 55° C., of more than 60° C., of more than 65° C., of more than 70° C., of more than 80° C., of more than 90° C., of more than 100° C.

In some embodiments, the core comprises a network-structured polymer encapsulating the active agent within the network. In some embodiments, the core comprises a network-structured polymer encapsulating the active agent, wherein the active agent is a liquid (e.g., oil). In some embodiments, the active agent is in a form of droplets. In some embodiments, the active agent is in a form of a continuous phase within the core. In some embodiments, the active agent is dispersed within the polymeric matrix. In some embodiments, the active agent is dispersed within the core.

In some embodiments, the active agent comprises a water-soluble molecule, a lipophilic molecule, a water-insoluble molecule. In some embodiments, the water-soluble molecule has solubility in an aqueous solvent of more than 10 g/L. In some embodiments, the active agent comprises an essential oil, an herbicide, a pesticide, a fungicide, or any combination thereof.

As used herein, the term “active agent” refers to any type of material that can be encapsulated in the core and retain plant protective qualities. In some embodiments, the active agent has anti-fungal, anti-microbial, anti-insect, anti-viral, anti-mold, or plant protective qualities. In some embodiments, the active agent functions as a pesticide. In some embodiments, the active agent comprises a pesticide, an herbicide, a fragrance, a fungicide or any combination thereof. In some embodiments, the active agent comprises a plurality of active agents, wherein the active agents are as described herein.

In some embodiments, the active agent is lipophilic. In some embodiments, the active agent is an essential oil. In some embodiments, the essential oil is a thymol. In some embodiments, the essential oil is a carvacrol. In some embodiments, the active agent is a mixture of thymol and carvacrol. In some embodiments, the active agent is a combination of more than one essential oil.

In some embodiments, the composition of the invention is characterized by adhesiveness or tackiness to the plant, and/or to a part of the plant. In some embodiments, the composition of the invention is characterized by adhesiveness to a plant tissue. In some embodiments, the composition of the invention is characterized by adhesiveness to a plant leaf.

In some embodiments, the composition of the invention is stably adhered to the plant, and/or to a part of the plant. In some embodiments, the composition of the invention remains stably bound (physically stable) or in contact with the plant, and/or with a part of the plant for a time period of between 1 day and 6 months, between 1 day and 1 months, between 1 day and 10 days, between 10 days and 20 days, including any range between. In some embodiments, the composition of the invention remains stably bound (physically stable) or in contact with the plant, and/or with a part of the plant when exposed to ambient conditions at the area under cultivation (e.g., exposure to UV light, rain, moisture, temperatures of between 0 and 50° C., etc.).

In some embodiments, the terms “composition” and “emulsion” are used herein interchangeably.

In some embodiments, the composition of the invention is configured to deactivate or kill an insect or aphid by immobilizing thereof.

In some embodiments, the composition is for use as: an anti-fungal coating, an anti-microbial coating, an anti-insect coating, an anti-viral coating, an anti-mold coating, a plant protective coating, or a pesticide coating. In some embodiments, the composition is for use as an insecticidal composition.

In some embodiments, upon application of the composition of the invention to a plant and/or a part thereof (e.g., a leaf), a coating layer is obtained. In some embodiments, the coating layer covers at least 50%, at least 70%, at least 90%, at least 80%, of the leaf surface area, including any range between.

In some embodiments, the coating layer is stably adhered to the plant and/or a part thereof. In some embodiments, stably adhered refers to the capability of the coating layer to substantially retain on the surface the plant and/or a part thereof for a time period up to 60 days (d), up to 30 d, up to 25 d, up to 20 d, up to 15 d, up to 10 d, including any range between.

In some embodiments, the coating layer is characterized by a tackiness sufficient for entrapping, immobilizing and/or killing an aphid. In some embodiments, the coating layer is characterized by a sufficient tackiness for a time period up to 60 days (d), up to 30 d, up to 25 d, up to 20 d, up to 15 d, up to 10 d, including any range between.

### The Article

According to some embodiments, the present invention provides an article comprising the emulsion of the present invention. In some embodiments, the article comprises the emulsion and a substrate, wherein the emulsion is in the form of a coating layer on the substrate. In some embodiments, the emulsion is in the form of a coating layer in at least a portion of a surface of the substrate. In some embodiments, the substrate is a plant or a part of the plant.

In some embodiments, there is an insecticidal composition comprising an effective amount of the composition of the invention. In some embodiments, effective amount is sufficient to induce an insecticidal effect. In some embodiments, effective amount refers to a concertation of the core-shell particles of the invention within the insecticidal composition. In some embodiments, effective amount is or comprises an insecticidal effective amount. In some embodiments, effective amount is sufficient for forming a coating layer on top of the plant, wherein the coating layer has sufficient adhesiveness to the pest, so as to at least partially deactivate the pest (e.g., an insect and/or an aphid).

In some embodiments, the insecticidal composition optionally comprises an agriculturally acceptable carrier. In some embodiments, the insecticidal composition is formulated for spraying. In some embodiments, the insecticidal composition comprises (C1-C4) alkylsilyl modified silica or a fluorinated silica (as the hydrophobic metal oxide nanoparticle), polyacrylate as the hydrophilic polymer, and a plant oil as the major phase.

In some embodiments, the composition and/or the insecticidal composition of the invention is for use as an insecticide. In some embodiments, the composition and/or the insecticidal composition of the invention is for reduction of pest loading on cultivating plants or at the area under cultivation, wherein pest comprises an insect and/or an aphid.

In some embodiments, a coating layer comprising the composition and/or the insecticidal composition in contact with or adhered to a plant and/or a part thereof, is stable to climatic changes. In some embodiments, the coating layer is stable to temperature changes, heat, cold, UV radiation and atmospheric corrosive elements. In some embodiments, the insecticidal properties and/or tackiness of the coating layer are not affected or altered by climatic changes as described herein.

In some embodiments, the article according to the present invention, is stable to climatic changes. In some embodiments, the article is stable to temperature changes, heat, cold, UV radiation and atmospheric corrosive elements. In some embodiments, the insecticidal properties and/or tackiness of the article are not affected or altered by climatic changes as described herein.

### The Method

In another aspect, there is provided a method for controlling a pest or reducing growth thereof, comprising providing the insecticidal composition of the invention; and contacting an insecticidal effective amount of the insecticidal composition with a locus infested with the pest.

The term “locus” as used herein, means a habitat, plant, seed, material, or environment, in which a pest is growing, may grow, or may traverse. For example, a locus may be: crops, trees, fruits, cereals, fodder species, vines, and/or ornamental plants, the interior or exterior surfaces of buildings (such as places where grains are stored); the materials of construction used in buildings (such as impregnated wood). In some embodiments, the terms “locus” and “area under cultivation” are used herein interchangeably. In some embodiments, the terms “locus” refers to a plant and/or to a part of the plant (e.g., a leaf).

In some embodiments, there is a method for controlling a pest or reducing growth thereof, comprising applying an effective amount of the insecticidal composition of the invention to at least a portion of a plant, or an area under cultivation infested with the pest, thereby controlling or reducing growth of the pest.

In some embodiments, the insecticidal effective amount is or comprises application of between 0.1 and 100 L, between 0.1 and 0.5 L, between 0.5 and 1 L, between 1 and 2 L, between 0.1 and 10 L, between 1 and 10 L, between 2 and 5 L, between 5 and 10 L, between 1 and 50 L, between 10 and 100 L, between 10 and 50 L, between 50 and 100 L of the composition (or emulsion) of the invention, including any range between; wherein the composition/emulsion is applied per 1 ha of the cultivated area.

One skilled in the art will appreciate that the exact effective amount may vary depending inter alia on a plant specie, plant density within the cultivated area, specific climatic conditions, application time, etc. As exemplified herein, different plant species require application of different effective amount of the composition/emulsion of the invention.

In some embodiments, the method is for killing a plant pathogen or for reducing plant pathogen load. In some embodiments, the method is for killing a pathogen or reducing growth thereof by administering to a plant the composition described hereinabove.

In some embodiments, the pest is a pathogenic parasite. In some embodiments, the pathogen is an insect. In some embodiments, the pathogen is an aphid selected from, but is not limited to: Myzus persicae, Aphis gossypii, Brevicoryne brassicae, Aphis nerii, Bemisia tabaci and Rhopalosiphum maidis. According to some embodiments of the present invention, the disclosed compositions are for use in the reducing growth or for complete inhibition of a pathogen.

In some embodiments, upon application of the composition of the invention to a plant and/or a part thereof (e.g., a leaf), a coating layer is obtained. In some embodiments, the coating layer covers at least 50%, at least 70%, at least 90%, at least 80%, of the leaf surface area, including any range between.

In some embodiments, the coating layer is stably adhered to the plant and/or a part thereof. In some embodiments, stably adhered refers to the capability of the coating layer to substantially retain on the surface the plant and/or a part thereof for a time period up to 60 days (d), up to 30 d, up to 25 d, up to 20 d, up to 15 d, up to 10 d, including any range between.

In some embodiments, the coating layer is characterized by a tackiness sufficient for entrapping, immobilizing and/or killing an aphid. In some embodiments, the coating layer is characterized by a sufficient tackiness for a time period up to 60 days (d), up to 30 d, up to 25 d, up to 20 d, up to 15 d, up to 10 d, including any range between.

In some embodiments, the term “reducing”, or any grammatical derivative thereof, indicates that at least 20%, 30%, 40%, 50%, 60%, 70%, 80%, 90%, or more, reduction of growth or even complete growth inhibition in a given time as compared to the growth in that given time of the pathogen not being exposed to the treatment as described herein. In some embodiments, the term “completely inhibited”, or any grammatical derivative thereof, refers to 100% arrest of growth in a given time as compared to the growth in that given time of the pathogen not being exposed to the treatment as described herein. In some embodiments, the terms “completely inhibited” and “eradicated” including nay grammatical form thereof, are used herein interchangeably.

In some embodiments, the insecticidal composition of the invention is characterized by adhesiveness to at least a part of the plant.

In some embodiments, the plant comprises a cultivating plant or a part thereof.

In some embodiments, the insecticidal composition of the invention is applied to a plant or to a part of a plant via a method comprising: immersion, coating, irrigating, dipping, spraying, fogging, scattering, painting, injecting, or any combination thereof. In some embodiments, the plant or plant part is pre-harvested. In some embodiments, the method is directed to storing a plant or plant part, comprising exposing a post-harvest plant or plant part to an effective amount of the composition.

### General

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

### Materials & Methods

The inventors successfully utilized alkylated (or alkylsilyl-modified) silica nanoparticles for the preparation of the exemplary compositions of the invention as described herein. The inventors used Aerosil R816 silica modified with hexadecyl silane, (particle size of about 12 nm), and Aerosil R812 silica modified with hexamethyldisilazane, i.e., trimethyl-silyl modified silica particles.

### Miscibility of Oil Pairs

Equal volumes (5 ml) of a canola oil and water were placed in a screw-cap glass vial. The mixture was sonicated for 10 min using an ultra-sonication at 25% amplitude, and the volume of the two oil phases separated after a few minutes was measured.

### Preparation of Polymer Solutions

The poly(acrylic acid sodium salt) was weighed (according to the polymer concentration at 0.5%) and uniformly dispersed in water (100 mL).

### Preparation of Tackifier Solutions

The tackifier (Glycine) was weighed (according to the concentration at 1%) and uniformly dispersed in water (100 mL).

### Preparation of Water-In-Oil Emulsions

The required mass of silica nano particles (1 wt %) was placed in the vial, followed by the addition of canola oil (5 ml). The required volume (5 ml) of water (0.5% poly(acrylic acid sodium salt)) was then added. The mixture was sonicated for 10 min using an ultra-sonication at 25% amplitude.

### Preparation of Oil-In-Water Emulsions

The required mass of silica nano particles (1 wt %) was placed in the vial, followed by the addition of water (6-8 ml, (0.5% poly(acrylic acid sodium salt)). The required volume (2-4 ml) of oil was then added. The mixture was sonicated for 10 min using an ultra-sonication at 25% amplitude.

Preparation of Oil-In-Water Emulsions with a Tackifier

The required mass of silica nano particles (1 wt %) was placed in the vial, followed by the addition of water (6-8 ml with 1% tackifier). The required volume (2-4 ml) of oil was then added. The mixture was sonicated for 10 min using an ultra-sonication at 25% amplitude.

Preparation of Emulsions with Active Materials

The required mass of silica nano particles (1 wt %) was placed in the vial followed by the addition of canola oil (5 ml), Carvacol (0.1, 0.2, 0.4, 0.5%) and thymol (0.1, 0.2, 0.4, 0.5%). The required volume (5 ml) of water (0.5% poly(acrylic acid sodium salt)) was then added. The mixture was sonicated for 10 min using an ultra-sonication at 25% amplitude.

### Emulsion Characterization

### Confocal Laser Scanning Microscopy (CLSM) Analysis

Images were acquired using a Leica SP8 laser scanning microscope (Leica, Wetzlar, Germany), equipped with a solid state laser with 488 nm light, HC PL APO CS 20×/0.75 objective (Leica, Wetzlar, Germany) and Leica Application Suite X software (LASX, Leica, Wetzlar, Germany). The Nile Red signal was imaged using a solid-state laser with 552 nm light, and the emission was detected in the range of 580-670 nm. For the analysis, 5 μL were taken from the emulsion and drop-cast on a microscopic slide, equipped with a coverslip.

### Confocal Microscopy Characterization

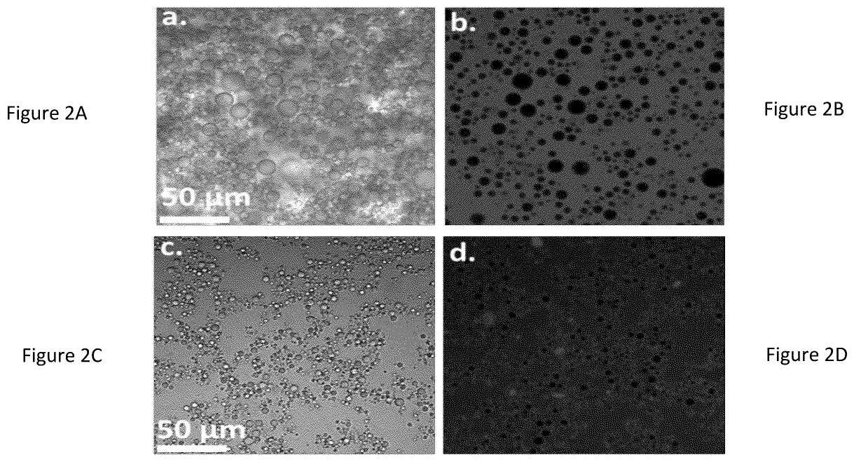

The confocal microscopy characterization of the emulsions revealed the successful formation of W/O Pickering emulsion (FIG. 2). To distinguish between the two phases, Nile red, a florescent dye soluble in canola oil but insoluble in water was added to the emulsions. Confocal microscopy analysis of the emulsions after addition of Nile red showed that the continuous phase and silica were selectively labelled with Nile red according to their typical excitation of red color. Hence the major phase of the emulsions is canola oil; i.e., the target composition is water in canola oil Pickering emulsions. The water phase micro droplets were also observed dispersed throughout the canola oil continuous phase. The confocal microscopy results also confirm that silica is the stabilizing agent of the emulsions.

### Example 1

### Insecticidal Activity of an Exemplary Emulsion of the Invention

Exemplary insecticidal composition of the invention comprising (i) 1% Silica, canola oil:water (1:1); (ii) 1% Silica, canola oil:water (1:1), 0.5% Carvacol, 0.5% Thymol; and (iii) % Silica, canola oil:aqueous solution (1:1), wherein aqueous solution comprised 0.5% by weight of poly(acrylic acid) sodium salt, were successfully applied on potato leaves by spraying. Subsequently, the number of immobilized (killed) insects (white fly) and aphids has been evaluated and scored. The tested compositions exhibited solid insecticidal activity.

Table 1 represents the result of an exemplary experiment, where the composition (i) has been applied to the infested potato leaves by spraying.

TABLE 1
Number of whiteflies and aphids were trapped and killed on the
leaves surfaces upon application of an exemplary W/O emulsion
(1% Silica such as Aerosil R816/812, canola oil:water (1:1)).

| Number of | Number of | Number of |
|---|---|---|
| leaves | White fly | aphids |
| 1 | 41 | 12 |
| 2 | 19 | 11 |
| 3 | 14 | 6 |
| 4 | 29 | 11 |
| 5 | 60 | 08 |
| 6 | 15 | 10 |
| 7 | 9 | 26 |
| 8 | 26 | 21 |
| 9 | 22 | 06 |
| 10 | 15 | 12 |
| 11 | 42 | 05 |

Furthermore, the inventors tested the emulsions described herein on various plants. Various long term aphid protection was obtained for different plant species upon application of the composition of the invention (with or without Carvacol/Thymol). Additionally, no substantial phytotoxicity has been observed upon application of the emulsions of the invention on the plants. The results of application of the exemplary W/emulsion of the invention on various plants are summarized in Tables 2-3 below.

TABLE 2
insect protection on cucumber plants (Myzus persicae)
upon application of an exemplary W/O emulsion

| | | | | | Attract |
|---|---|---|---|---|---|
| | | | | | towards |
| | | | | Duration | insect |
| | | Plant name, | | of aphid | behaviour |
| | | number of | Active | protection | per leaf |
| # | Formulation | plants | materials | (days) | (Trap) |
| 1 | Sample-1 | C. sativus, | 1% Carvacol | 8 | 3 ± 4 |
| | | 17-19 plants | 1% Thymol | | |
| 2 | Sample-2 | C. sativus, | 0.5% Carvacol, | 8 | 2 ± 2 |
| | | 17-19 plants | 0.5% Thymol | | |
| 3 | Sample-3 | C. sativus, | Without active | 8 | 1 ± 1 |
| | | 17-19 plants | materials | | |

TABLE 3
insect protection on potato plants upon application of an exemplary W/O emulsion

| | | | | Duration of | Attract towards insect | |
|---|---|---|---|---|---|---|
| | | | | whitefly/aphid | behaviour per leaf (Trap) | |
| | | Active | Phyto- | protection | | Aphid & other |
| # | Formulation | materials | toxicity | (days) | Whitefly | insects |
| 1 | Sample-1 | 0.4% | — | 7 | 16 ± 9 | 6 ± 5 |
| | | Carvacol, | | | | |
| | | 0.4% | | | | |
| | | Thymol | | | | |
| 2 | Control | Without | — | 7 | 47 ± 6 | 10 ± 6 |
| | | active | | | | |
| | | materials | | | | |

Additionally, exemplary O/W emulsions has been successfully tested by the inventors. O/W emulsions were characterized by fast absorption into the plant tissue (about 15 min). Furthermore, the tested emulsions exhibited insect trapping capabilities on both front side-, and back side of the leaf.

The inventors successfully implemented various O/W emulsions comprising canola oil/pal oil water ratios between 2:8 and 4:6. Additional O/W emulsions comprising water immiscible oils (as the minor phase), such as mineral oil, and/or fatty acids or lipids currently undergo plant studies.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An emulsion comprising a major phase and a plurality of core-shell particles, wherein:
a. each core-shell particle comprises a core and a shell stabilizing the core;
b. said shell comprises hydrophobic metal oxide nanoparticles and a hydrophilic polymer in contact with said hydrophobic metal oxide nanoparticles;
c. said major phase and said core comprise immiscible liquids selected from an aqueous solution, or an oil respectively;
d. a weight per weight (w/w) concentration of said hydrophobic metal oxide nanoparticles within said emulsion is between 0.001 and 10%;
e. a w/w ratio between the aqueous solution and the oil within the emulsion is between 10:1 and 1:10.

2. An emulsion comprising a major phase and a plurality of core-shell particles, wherein:
a. each core-shell particle comprises a core, and a shell comprising hydrophobic metal oxide nanoparticles stabilizing the core;
b. said shell comprises hydrophobic metal oxide nanoparticles and a hydrophilic polymer in contact with said hydrophobic metal oxide nanoparticles said major phase is or comprises an aqueous solution;
c. and wherein a weight per weight (w/w) concentration of said hydrophobic metal oxide nanoparticles within said emulsion is between 0.001 and 10%;
d. a w/w ratio between the major phase and the oil within the emulsion is between 10:1 and 1.2:1.

3. An emulsion comprising a major phase and a plurality of core-shell particles, wherein:
a. each core-shell particle comprises a core and a shell stabilizing the core;
b. said core comprises an aqueous solution,
c. said shell comprises hydrophobic metal oxide nanoparticles and a hydrophilic polymer in contact with said hydrophobic metal oxide nanoparticles;
d. said major phase comprises an oil; and wherein a weight per weight (w/w) concentration of said hydrophobic metal oxide nanoparticles within said emulsion is between 0.001 and 10%.

4. The emulsion of claim 1, wherein said hydrophilic polymer is water miscible or water dispersible.

5. The emulsion of claim 1, a w/w ratio between said hydrophilic polymer and said hydrophobic metal oxide nanoparticles within the emulsion is between 5:1 and 1:5.

6. The emulsion of claim 1, wherein said hydrophilic polymer comprises a polyanion, a polycation, a polyol, including any mixture or a copolymer thereof.

7. The emulsion of claim 1, wherein said hydrophobic metal oxide nanoparticles comprises chemically modified metal oxide nanoparticles.

8. The emulsion of claim 7, wherein said chemical modification comprises any of (C1-C20)alkyl, (C1-C4)alkylsilyl, (C4-C20)alkylsilyl, (C1-C20)alkylsilyl, phenyl, thiol group, vinyl, fluoroalkyl, haloalkyl, halogen, epoxy, a cycloalkane, an alkene, a haloalkene, an alkyne, an ether, a silyl group, a siloxane group, and a thioether or any combination thereof.

9. The emulsion of claim 1, wherein said metal oxide comprises nanoclay, SiO₂, TiO₂, Al₂O₃, Fe₂O₃, ZnO, and ZrO or any combination thereof.

10. The emulsion of claim 1, wherein said core-shell particle has a particle size of 0.5 μm to 500 μm.

11. The emulsion of claim 1, wherein said shell has a thickness of 10 nm to 100 μm.

12. The emulsion of claim 1, wherein said hydrophilic polymer comprises any one of a polyacrylate, an acrylate polymer, carboxymethyl cellulose (CMC), alginate, pectin, chitosan, polymetacrylate, polyvinyl alcohol (PVA), including any salt, any protonated form thereof, any copolymer or any combination thereof.

13. The emulsion of claim 1, wherein a w/w concentration of said hydrophilic polymer within said emulsion is between 0.001 and 15%.

14. The emulsion of claim 3, wherein a w/w ratio between said major phase and said aqueous solution within said emulsion is between about 100:1 and 1:1.

15. The emulsion of claim 1, wherein said oil comprises a vegetable oil, a mineral oil, a saturated fatty acid, an unsaturated fatty acid, and a lipid, or any combination thereof.

16. The emulsion of claim 1, wherein said hydrophilic polymer is immiscible with said oil.

17. (canceled)

18. The emulsion of claim 1, wherein said emulsion comprises between 0.1 to 5% of an active agent selected from the group comprising a pesticide, an herbicide, an insecticide, and an insect attracting agent, or a combination thereof, optionally wherein said active agent comprises an essential oil.

19. (canceled)

20. (canceled)

21. The emulsion of claim 1, wherein said emulsion has adhesiveness to a plant, a part of a plant, a plant tissue, a leaf, or any combination thereof.

22. An insecticidal composition comprising an effective amount of the emulsion of claim 1, optionally comprising an agriculturally acceptable carrier.

23. (canceled)

24. The insecticidal composition of claim 22, wherein said hydrophobic metal oxide nanoparticle comprises (C1-C20)alkylsilyl modified silica, wherein said hydrophilic polymer comprises polyacrylate, and wherein said oil comprises plant oil.

25.-30. (canceled)
